(19) United States
(12) Patent Application Publication
(10) Pub. No.: US 2016/0051149 A1
(43) Pub. Date: Feb. 25, 2016

(54) Photoacoustic Probe for Burn Injury Diagnosis

(71) Applicant: THE CURATORS OF THE UNIVERSITY OF MISSOURI, Columbia, MO (US)

(72) Inventor: John Andrew Viator, Columbia, MO (US)

(21) Appl. No.: 14/780,084

(22) PCT Filed: Apr. 18, 2014
(86) PCT No.: PCT/US14/34693

§ 371 Date: Sep. 25, 2015

Related U.S. Application Data
(60) Provisional application No. 61/814,679, filed on Apr. 22, 2013.

(51) Int. Cl. A61B 5/00

(52) U.S. Cl. CPC A61B 5/0095; A61B 5/445; A61B 5/6801; A61B 5/7278; A61B 5/7203; A61B 2562/0204

(57) ABSTRACT

A method and apparatus for depth profiling the structure of a subsurface region of skin, in particular burned skin, wherein the method/apparatus comprises directing laser light at an absorbing target to generate ultrasonic sound waves, which are used to determine the sound speed in the skin, and using the determined sound speed in conjunction with two-wavelength photoacoustics to depth profile of the structure of the subsurface of the skin.

### CROSS REFERENCE TO RELATED APPLICATIONS

This application claims the benefit of U.S. Provisional Application No. 61/814,679 filed Apr. 22, 2013, which is hereby incorporated herein by reference in its entirety.

### FIELD OF THE INVENTION

The invention is related to the field of noninvasive depth profiling of skin parameters using a photoacoustic probe and in particular to depth profiling skin burns.

### BACKGROUND OF INVENTION

There are an estimated 500,000 cases of burn injury that require medical attention in the United States every year, with 45,000 requiring hospitalization. This number gives rise to an estimated $4 billion annual cost. Half of these hospitalizations are at one of 125 regional burn centers. The percentage admitted to burn centers has increased steadily in recent decades, with growing recognition of the special needs of burn patients and continuing advances in the technical resources and skills of those who refer, transport, and treat them.

Early and accurate determination of burn depth is crucial in deciding which steps are taken to treat a burn wound. Currently, clinical observation, is the standard method for determining burn depth. Although it is an accurate predictor of full-thickness burns, it is only about accurate about one-half the time in the diagnosis of partial thickness burns. Many methods proposed for burn depth determination simply attempt to ascertain if the injury will heal within 3 weeks, as wounds that spontaneously heal within that period usually do so without scarring or impairment. Wounds that take longer to heal require surgical intervention to prevent complications. An accurate depth determination, however, would not only give an indication of the healing potential, but also aid the burn surgeon in the assessment of debridement depth, if warranted. If depth profiles of the wounds were available, the burn surgeon would be able to more accurately determine whether tissue is necrotic, reversibly damaged, or viable. Necrotic tissue must be debrided, while reversibly damaged tissue, overlying normal, viable tissue, should be allowed to heal. Preferably, debridement is performed as early as possible to allow for more rapid wound closure, prevention of infection, and thus, a shortened hospital stay.

The three tissue conditions noted above have contrasting optical properties, leading one to believe that an optical probing method might be useful for burn depth profiling. Unfortunately, optical signals degrade quickly in human skin owing to its highly scattering nature, which limits the information that may be determined. For example, Laser Doppler Imaging (LDI) only provides information about blood perfusion from the surface and gives no depth or imaging information. Optical coherence tomography (OCT), however, has been used to provide detailed images but it is limited to less than 1.5 mm depth, which is insufficient to image skin, which may be up to 5 mm deep.

While optical methods for probing burn depth will be hampered due to photon scattering by tissue, acoustic wave propagation in tissue is largely unaffected by acoustic scattering. So, acoustic waves tend to travel through layered tissue with very little signal degradation. It is this propagation environment that allows for conventional ultrasound imaging. Ultrasound has been used to study depth of burn injury but ultrasound methods depend upon on the ability to detect damage in the deep dermal capillary plexus. The result is not an exact measure of burn depth, but an estimate of whether the injury required surgical intervention or not.

Photoacoustic devices and methods (such as disclosed in U.S. Pat. No. 7,322,972, Viator et al., which is incorporated by reference herein in its entirety) have been used to provided depth and imaging information of the full thickness of skin, however, it has been determined by the inventors hereof that the accuracy of the depth information may be improved.

There is a need for an apparatus and/or method for more accurately determining the extent of burn damage—particularly, that associated with partial thickness burns. Such an apparatus and/or method would likely be a valuable tool in the diagnosis, monitoring, and treatment of burn wounds by clinicians. For example, accurate depth profiling of a burn wound is likely to allow necrotic tissue to be differentiated from reversibly damaged or viable tissue, thereby making early and accurate excision of the burn wound possible, which is often an important aspect of the treatment of partial thickness burns. Further, such guided precision may allow for increased preservation of subsurface epithelial structures that a responsible for healing. Also, such information may also be important when deciding whether to utilize artificial skin in the treatment of burn patients.

### SUMMARY OF INVENTION

In one embodiment, the present invention is directed to an apparatus for depth profiling the structure of a subsurface region of skin, the apparatus comprising: (a) at least one light source for generating a light pulse at a wavelength and intensity effective to generate photoacoustic responses in (i) an absorbing target and (ii) the subsurface region of the skin; (b) at least one optic fiber coupled to the at least one light source for delivering the light pulse to the absorbing target and the skin; (c) a probe that comprises (i) a housing comprising an exterior surface, wherein the exterior surface at least a portion of which is placed in contact with the skin, (ii) an inner chamber disposed within the housing and defined, at least in part, by an interior surface of the housing opposite the portion of the exterior surface placed in contact with the skin, (iii) a terminus of the at least one optic fiber located such that it is within or it defines, at least in part, the inner chamber, (iv) the absorbing target, which is located proximate to or in contact with said interior surface of the inner chamber, wherein the photoacoustic response of the absorbing target produces ultrasonic waves, at least a portion of which propagate into and are reflected by the skin, and (v) an acoustic detector for receiving and generating electrical signals in response to the reflected ultrasonic waves and photoacoustic waves generated by the photoacoustic response in the subsurface region of the skin, wherein the acoustic detector is within or defines, at least in part, the inner chamber and is spaced apart from said interior surface of the inner chamber to provide an acoustic delay greater than a delay of electrical noise arising from the light pulse to prevent contamination of said electrical signals; and (d) at least one circuit coupled to the acoustic detector for processing said electrical signals, wherein the electrical signals generated in response to the reflected ultrasonic waves are interpretable as sound speed in the skin and the electrical signals generated in response to the photoacoustic waves are interpretable, in conjunction with the sound speed, as a depth profile of the structure of the subsurface region of the skin.

In another embodiment, the present invention is directed to a method of depth profiling the structure of a subsurface region of skin, the method comprising: (a) generating a light pulse from at least one light source and directing the light pulse through at least one optic fiber coupled to the at least one light source to deliver the light pulse to and generate photoacoustic responses in: (i) an absorbing target producing ultrasonic waves, at least a portion of which propagate into and are reflected by the skin; and (ii) the subsurface region of the skin producing photoacoustic waves in the subsurface region of the skin; (b) generating electrical signals with an acoustic detector in response to the reflected ultrasonic waves and the photoacoustic waves, wherein the acoustic detector is spaced sufficiently away from the skin to provide an acoustic delay greater than a delay of electrical noise arising from the light pulse to prevent contamination of said electrical signals; and (c) processing the electrical signals with at least one circuit coupled to the acoustic detector, wherein the electrical signals generated in response to the reflected ultrasonic waves are interpreted to determine a sound speed in the skin and the electrical signals generated in response to the photoacoustic waves are interpreted, in conjunction with the sound speed, to determine a depth profile of the structure of the subsurface region of the skin.

### BRIEF DESCRIPTION OF THE DRAWINGS

FIG. 1 is a schematic diagram of a scanning system showing coagulated blood being distinguished from viable blood and the depth of these structures being determined.

FIG. 2 are graphs showing the classification of a tissue phantom with viable and coagulated blood.

FIG. 3 is a schematic diagram of a photoacoustic probe embodiment of the present invention in which the left half of the laser beam is used to create an acoustic pulse that will be used to determine the speed of sound in the tissue and the right half of the laser beam is used to generate photoacoustic waves in the tissue using viable and coagulated blood for optical contrast.

FIG. 4 is a schematic diagram of the system used for burn depth.

### DETAILED DESCRIPTION OF INVENTION

The present invention is directed to an apparatus and method for depth profiling of tissue and/or tissue phantoms or simulations, including burned tissue and/or tissue phantoms comprising simulated burn damage, wherein the apparatus comprises a sensor that uses laser light to induce photoacoustic responses at (a) the skin surface in order to introduce an acoustic pulse that will be used to determine the acoustic environment (i.e., laser-induced ultrasonic pulses for determining acoustic properties) and (b) within the skin to provide a depth profile of burn injury (i.e., photoacoustic depth profiling).

This new type of sensor is referred to herein as a “dual sensor” and may be used to provide more accurate burn depth profiling than may be provided with conventional technologies. For example, conventional photoacoustic equipment and methods require or are based on an assumption that disregards the variable acoustic properties of skin. This is particularly disadvantageous when making burn depth profiles/measurements because sound speed changes when collagen tissue is denatured due to thermal insult. The disadvantage has been overcome, at least in part, by using laser-induced ultrasonic pulses to determine acoustic properties (e.g., sound speed) of each particular tissue being evaluated thereby enabling more accurate burn depth profiling using photoacoustic pulses.

One embodiment of the present invention is directed to an apparatus and method for performing depth profiling and imaging of burn injuries. It is envisioned that such an apparatus may comprise a pulsed laser system, detection and interface electronics, a hand piece, and display and be similar in appearance and operation to a portable ultrasound unit in that in operation a hand piece may be placed onto skin and the detected acoustic signals may be used to produce depth profile and/or image information that may be displayed, for example, on a monitor. With such information, a clinician/operator may be able to delineate regions of healthy and necrotic tissue, and intermediate layers, where tissue may recover or become necrotic. A clinician/operator may consider such information/images when evaluating whether excision of tissue may be needed during initial and/or continued diagnosis and treatment over an extended period. Such objective information about wound resuscitation is not currently available and may allow for accurate and repeatable burn injury diagnosis by physicians that may not be highly experienced in burn care. Thus, reliance upon and transport of patients to burn centers may be reduced.

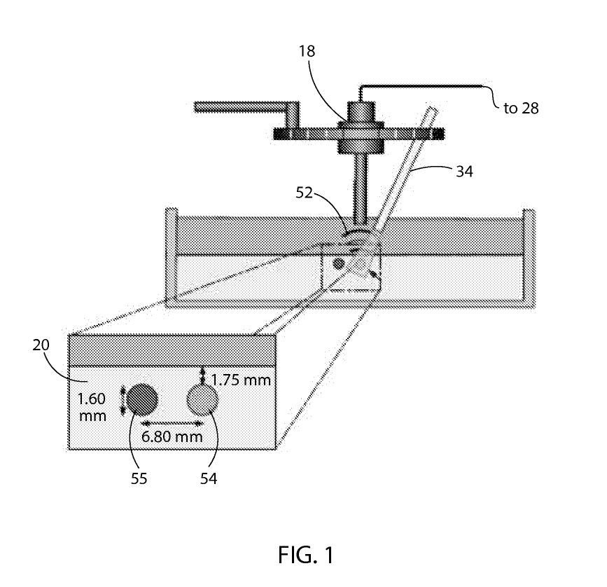

Referring to FIGS. 1, 3 and 4, an embodiment of an apparatus of the present invention comprises a photoacoustic probe 10 for non-invasive measurement of burn depth. The photoacoustic probe 10 comprises an optical fiber 34 (e.g., a 1500 μm diameter optical fiber) for laser light delivery and an acoustic transducer detector 18 (e.g., a piezoelectric polyvinylidene fluoride (PVDF), K-Tech, Albuquerque, N.M.) for acoustic detection in a housing/handpiece 40 of any desired shape and size and material (e.g., an acrylic cylindrical handpiece). The laser 30 may be Q-switched Nd:YAG laser operating at 532 nm with a 4 ns (FWHM) pulse duration (Quantel Brilliant, Big Sky Laser, Bozeman, Mont.). The laser output is focused into quartz fiber 34 (e.g., 1000 μm diameter resulting in a laser spot from the fiber of about 1.1 mm in diameter) which terminates in the photoacoustic probe 10. Laser energy may be monitored by an energy meter 36 prior to the fiber input. Output energy from the fiber 34 is selected to achieve the desired image and depth results while minimizing the possibility of additional tissue damage and/or patient discomfort (e.g., the energy output may be controlled within a range of about 1.5 to about 5 mJ). Although one optical fiber 34 is depicted, more than one optical fiber may be used. The number of optic fibers used is primarily a matter of design choice and may depend upon the amount of light intensity that is desired. It has been found that limiting the total amount of energy delivered to less than 22 mJ may be desirable to minimize patient discomfort. In such an embodiment, the multiple optical fibers are preferably configured to be coincident. Additionally, it is typically preferable for the fiber 34 to be arranged, configured, placed, situated, etc. to ensure mode mixing, which tends to increase the accuracy of radiant exposure measurements.

The transducer detector 18 within the probe 10 sends a signal via a cable 16 (e.g., a 1.1 mm diameter semi-rigid coaxial 10Ω cable available from Micro-Coax of Pottstown, Pa., product number UT-43-10) to an oscilloscope (not shown, e.g., a four channel digital oscilloscope such as the Tektronix TDS 3014 of Wilsonville, Oreg. having a bandwidth of 100 MHz and an input impedance of 1 MΩ, sampled at 1.25 GS/s, and triggered by a photodiode to monitor laser output) to convert the velocity potential to an actual pressure signal. The active area of the acoustic detector 18 may be selected as appropriate; for this particular embodiment it was 200 μm.

The probe 10 may be placed in contact with the target 20 (e.g., actual tissue or tissue phantoms) so that the laser-induced ultrasonic pulses 51 and photoacoustic waves 52 are generated below the acoustic detector 18. Thus, the optical fiber 34 irradiates an absorbing target 53 for generating ultrasonic sound waves (e.g., an acrylamide target from 3.5% acrylamide in water) and the tissue surface of target 20, inducing photoacoustic waves 52, which me be sensed in reflection mode by the acoustic detector 18. The absorbing target 53 may be of any material that absorbs laser light and closely matches the acoustic impedance of skin, including any number of phantom materials routinely used by ultrasound researchers. The photoacoustic waves may be generated, for example, by absorption of laser light in simulated viable blood vessel 54 and simulated coagulated blood vessel 55 within simulated tissue 20.

### Laser-Induced Ultrasonic Pulses for Determining Acoustic Properties

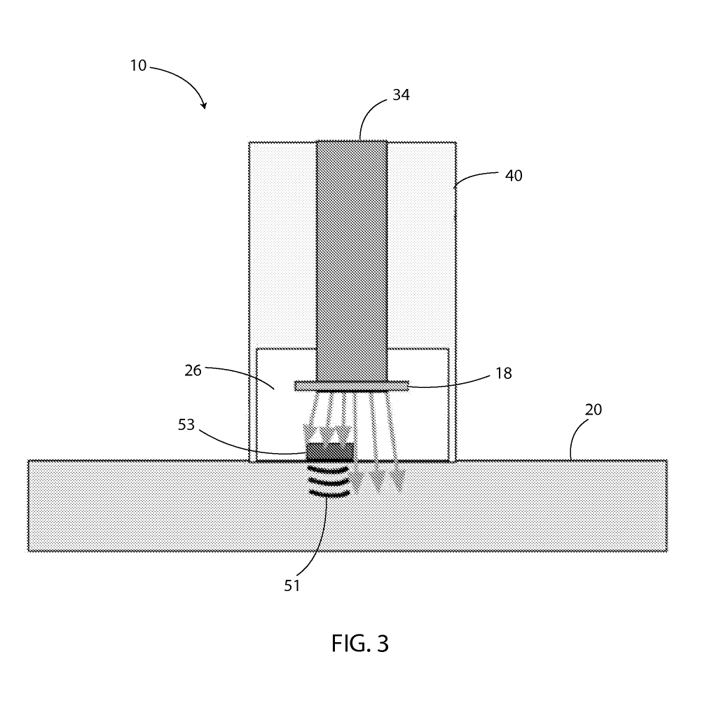

Referring to FIG. 3, the laser-induced acoustic waves 51 that are generated within the probe 10 are used to measure the acoustic impedance of the tissue (e.g., undamaged, viable or reversibly damaged, or necrotic). Specifically, the acoustic impedance is determined by the reflection of this acoustic energy. The acoustic impedance can then be used to determine the sound speed in the particular tissue being tested. More specifically, the sound speed may be determined by determining incident amplitude of the photoacoustic wave, which may be deduced from the laser pulse energy and the absorption coefficient of the absorbing target (e.g., acrylamide pad). The amplitude of the pressure wave is:

P=μₐ*Y*H₀

where p is the pressure amplitude, μₐ is the absorption coefficient of the pad, Y is 0.12 at room temperature, and H₀ is the energy per unit area of the laser beam. The reflected beam is given by:

R=(r₂*c₂−r₁*c₁)/(r₁*c₁ ^{-Fr}₂c₂)

where r₁ and r₂ are the densities of the pad and skin, respectively. The sound speeds of the pad and skin are c₁ and c₂, respectively. So R may be measured by detecting the acoustic wave that is reflected from the pad because rᵢ*cᵢ (i=1,2) is replaced with zᵢ, the acoustic impedance, which is the product of the sound speed and density, and z₁ is known because the speed and density of the pad is known. To solve for z₂, the acoustic impedance of the skin,

z₂=zᵢ*(R+1)/(R−1).

With the acoustic impedance of the skin and assuming density, the sound speed of the skin is determined. This determined sound speed is then used, instead of a pre-selected standard sound speed, in conducing the photoacoustic depth profiling and/or imaging (as described in greater detail below) conducted using the laser irradiation of the tissue from, for example, the other half of the laser energy from optical fiber 34 or from a different optical fiber (not shown).

### Photoacoustic Depth Profiling

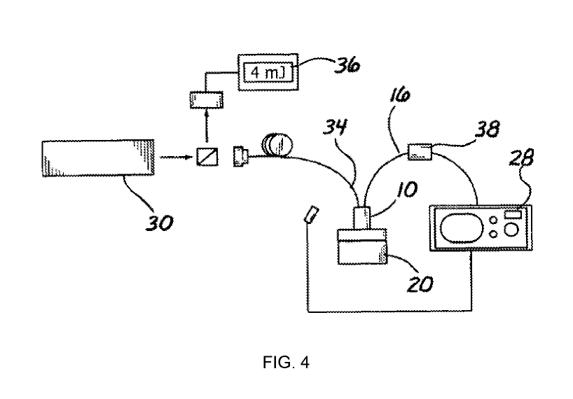

A portion of the fiber 34 and the detector 18 are in a water-filled chamber 26 defined in handpiece 40, which allows for acoustic impedance matching between the surface of target 20 and detector 18. The acoustic detector 18 is recessed (in this embodiment approximately 3 mm) into the probe housing 40 to separate the target surface from the detector 18. This separation creates an acoustic delay line of about 2 μs in order to prevent electrical noise caused by the laser pulse (occurring at 0-0.5 μs) from contaminating the photoacoustic signal that is transmitted via the coaxial cable 16 directly to (not pictured), or through an instrumentation amplifier 38 (e.g., having a gain of 125 such as available from Stanford Research Systems of Sunnyvale, Calif. model SR445) and to, an oscilloscope 28 as shown in FIG. 4. The raw photoacoustic signals from tissue phantoms/tissue may be de-noised using wavelet transforms and deconvolved with the probe's impulse response to give the approximate initial subsurface pressure distribution in tissue or tissue phantoms after the laser pulse.

Unlike optical methods, photoacoustic generation does not use photons as a signal, but as a means for delivering energy to subsurface blood vessels in the viable tissue underlying the thermally damaged layer. Once photon energy is absorbed (e.g., by hemoglobin), an acoustic wave is generated, which travels back to the skin surface where a detector measures acoustic wave shape and propagation time. The acoustic wave is a robust means for carrying information that is immune to the highly photon scattering and signal degrading nature of tissue. Thus, photoacoustics combines the high selectivity of optical absorption of targeted cells and tissue with the strong signal to noise ratio inherent in ultrasound propagation, the ideal balance of optical and acoustic techniques. Such data can be used to develop a depth map of the injured tissue.

More specifically, photoacoustic depth profiling of the invention uses pulsed laser irradiation to induce rapid thermoelastic expansion in targeted chromophores. This process is distinct from photoacoustic methods using modulated continuous wave irradiation, such as photoacoustic spectroscopy. Photoacoustic generation by thermoelastic expansion can be conceptually described as laser energy being quickly absorbed by a small volume such that resultant heating induces rapid expansion that manifests itself as a transient pulse of acoustic energy. Thermoelastic expansion occurs when the condition of stress confinement is achieved (i.e., where optical energy is deposited before the energy can propagate away acoustically). This condition is expressed as tₚ<δ/cₛ, here tₚ is the laser pulse duration, δ is the absorption depth of laser energy, and cₛ is the speed of sound in the medium. If the radiant exposure is not excessive, the resulting acoustic waves behave according to the linear wave equation. Furthermore, if the laser spot diameter is much larger than δ, then a simple plane wave analysis can be used, allowing acoustic propagation time to be used as an indicator of distance traveled. If stress confinement, linearity, and plane wave geometry are preserved, depth profiling and imaging of layered tissue may be achieved by simple photoacoustic analysis.

In order to classify photoacoustic signals arising from viable hemoglobin vs. coagulated blood, the method/apparatus uses the ratio of photoacoustic response at two laser wavelengths—green and red. As mentioned above, both wavelengths may be transmitted through the same fiber (simultaneously or in succession) or through multiple fibers. Viable hemoglobin, being red, responds strongly to green laser excitation, but poorly to red laser excitation. In contrast, coagulated blood is a broadband optical absorber, manifested by its brown color, and it responds moderately to both green and red laser excitations. So, a method/apparatus utilizing the ratio of photoacoustic response to green and red laser excitation will produce relatively large response values for viable hemoglobin and relatively low response values for coagulated blood.

Following the notation of Johson and Wichern (1998), it is stipulated that the photoacoustic ratios come from two distinct populations—π₁ representing the viable blood and π₂ representing the thermally coagulated blood. Using x to denote the ratio of photoacoustic response, f₁(x) and f₂(x) may be used to denote the probability density functions associated with the ratio of photoacoustic response. Further, the conditional probability of classifying a ratio as belonging to π₂ when it belongs to π₁ may be denoted by p(2|1) and whereas p(1|2) may be used to denote the converse (i.e., the conditional probability of classifying a ratio as belonging to 7₁ when it belongs to π₂). In this simplified analysis, equal costs of misclassification are assumed (e.g., misclassifying as viable may result in dead tissue not being excised, which may result in a bad tissue graft whereas misclassifying as coagulated may result in a removal of healthy tissue). Thus, for any classification rule, the average or expected cost of misclassification (ECM) is given by ECM =p(2|)p₁+p(1|2)p₂, where pᵢ (i=1,2) is the prior probability of πᵢ and p₁+p₂=1. A reasonable classification rule minimizes ECM and as a result ECM is given by the following:

$${R}_{1} \text{:} \frac{{f}_{1} ( x )}{{f}_{2} ( x )} \geq 1 , \\ {R}_{2} \text{:} \frac{{f}_{1} ( x )}{{f}_{2} ( x )} < 1$$

where it is assumed that p₁=p₂=½. In order to classify new measurements, the following may be used:

$$\frac{{( {x}_{0} - \overline{{x}_{2}} )}^{2} - {( {x}_{0} - \overline{{x}_{1}} )}^{2}}{2 {s}^{2}} \geq ln [ \frac{{p}_{2}}{{p}_{1}} ] ⇒ {x}_{0} ∈ {Π}_{1} , \\ \frac{{( {x}_{0} - \overline{{x}_{2}} )}^{2} - {( {x}_{0} - \overline{{x}_{1}} )}^{2}}{2 {s}^{2}} < ln [ \frac{{p}_{2}}{{p}_{1}} ] ⇒ {x}_{0} ∈ {Π}_{2} ,$$

where s² is the pooled variance. For a more detailed discussion regarding the foregoing, see Talbert et al., Photoacoustic discrimination of viable and thermally coagulated blood using a two-wavelength method for burn injury monitoring, Phys. Med. Biol. 52 (2007) 1815-1829, which is incorporated by reference herein in its entirety. In particular, see section 1. Statistical method for classifying coagulated and non-coagulated blood, of Talbert et al.

### Calibration of the Acoustic Detector

The acoustic detector 18 may be calibrated by conventional means by inducing photoacoustic waves in solutions where the absorption coefficient is known. For example, it has been calibrated by detecting photoacoustic waves in a transmission setup in which the free beam of the laser 30 was used because it provided a relatively large spot, which minimized diffraction and delivers more energy. The detector 18 was immersed in an absorbing solution and centered directly above the laser spot (4.6 mm in diameter). The radiant exposure was 0.084 J/cm², as calculated by measuring total energy with a standardized photodetector, (Molectron, Beaverton, Oreg.) and dividing by the spot size. The absorption coefficients of the solutions were 51, 103, 148, 197, and 239 cm⁻¹ at 532 nm. The equation

p(0)=½ΓH₀μₐ

was used to predict the photoacoustic pressure (J/cm³), where Γ is the Grueneisen coefficient, which models the fraction of optical energy converted to acoustic energy, and in this analysis Γ=0.12, μₐ is the absorption coefficient of the solution in cm⁻¹, and H₀ is the radiant exposure (J/cm²). The conversion 10 bar=1 J/cm³, was used to determine a calibration factor of mV/bar for the acoustic detector by dividing the amplitude of the acoustic waveform by the calculated pressure. The calibration factor was 1.31 mV/bar.

### Tissue Phantoms

The optical properties of the tissue phantoms are typically selected to mimic those of skin and/or burned skin. For example, it had been observed that 200-500 μm thick polyacrylamide layers make acceptable tissue phantoms. Polyacrylamide was chosen over collagen gels or agar as it can be made as thin as 50 μm and sets within minutes of adding a chemical initiator. The polyacrylamide tissue phantoms were made with 20% acryl amide in water with added dye for absorption and fat emulsion for scattering from skin to have optical properties similar to those of burned and viable skin. Specifically, Direct Red 81 (Sigma Chemical, St. Louis, Mo.) was used to simulate hemoglobin absorption and a 1% Intralipid (Abbott Laboratories, North Chicago, Ill.) solution was added to approximate 200 cm⁻¹, the approximate scattering coefficient of human skin at 532 nm. Phantoms of tissue may be prepared using three layers: a first layer to representing epidermis (e.g., about 200 μm thick with μₐ=25 cm⁻¹), an intermediate turbid layer representing necrotic tissue with no blood flow (e.g., which can be of different thickness depending upon simulated burn severity such as about 270, 330, 410, and 500 μm thick) formed by including the aforementioned Intralipid in the acrylamide so that μₛ=200, and an underlying layer representing perfused tissue (e.g., 1 mm thick) formed by including Intralipid so it is turbid and μₛ=200 cm⁻¹ and μₐ=25 cm⁻¹. To create layers acryl amide solutions may be injected between glass slides with plastic feeler gauge stock of various thicknesses used as spacers (Feeler gauge stock, McMaster-Carr, Los Angeles, Calif.).

### EXAMPLE

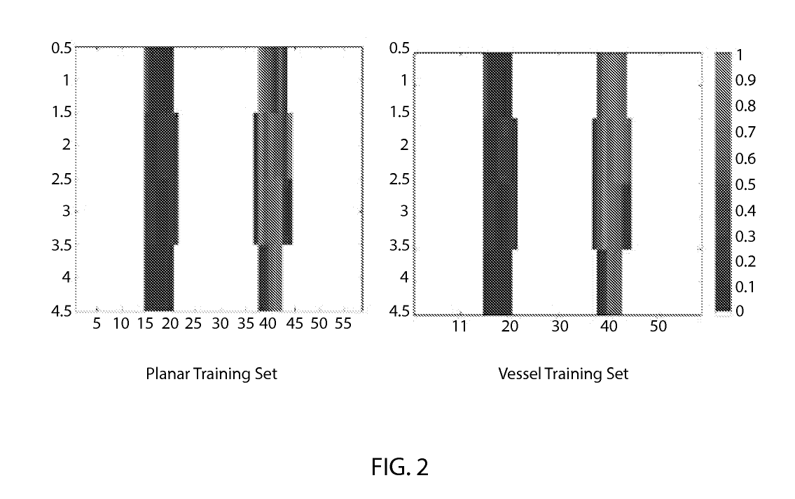

The above-described method for conducting photoacoustic depth profiling has been conducted to classify a tissue phantom comprising viable and coagulated blood as shown in FIG. 1. The classification results being displayed in in FIG. 2. The data depicted in the left graph was determined using a small cup-like container of blood, or coagulated blood having an acoustic sensor at the bottom of the container (i.e., an idealized configuration with a planar blood surface) whereas the right graph shows classification obtained using more realistic tubes containing blood.

### Prophetic Examples

### Characterization Using Tissue Phantoms

Tissue phantom experiments will be used to calibrate and test the limits of the photoacoustic system. Layered phantoms will be made ranging from 50 μm to 1 cm, with absorbing layers simulating burn layers. Additionally, absorbing spheres and cylinders will be embedded within turbid phantoms at precise depths for photoacoustic measurement.

Layered phantoms will be made constituting a burned layer over a layer of viable tissue. The viable tissue layer will be made 5 mm thick and will have scattering properties of normal skin. The absorption coefficient will be made to match a condition of 10% blood volume fraction, which represents inflamed tissue having a higher than normal hemoglobin content. Burn layers will be made with normal scattering properties and initially with no optical absorption. These layers will be made from 50-3000 μm thick in increments of 100 μm.

The photoacoustic probe will be placed directly on these phantoms and photoacoustic signals will be generated. The signals will be analyzed to check if the depth of the burn layer corresponds to the actual thickness of the phantom.

Imaging algorithms will be applied to the photoacoustic signals to determine whether irradiating a planar tissue phantom will result in an image of a plane.

The tests will be repeated with additional steps of complexity. For example, in one such increased complexity test, a cylinder of acrylamide dyed with Methylene Blue will be embedded within a burn layer to simulate a thrombosed vessel. The optical absorption of the Methylene Blue will be different from the absorption of the Direct Red used in the viable layer simulating hemoglobin. Several laser wavelengths will be used to irradiate the phantom and imaging will be performed. These wavelengths will target specific absorption peaks in Methylene Blue and Direct Red. The result should show distinct absorption by the thrombosed vessel and the deeper viable layer. Vessel size will be 100-500 μm, allowing for the actual thickness of the burned layer. In another such test, polyacrylamide spheres ranging from 300-1000 microns in diameter made from Methylene Blue will be embedded within the burned layer. These spheres will simulate coagulated blood and hemorrhage in the burned layer.

After using dyes, test will be performed using whole and heat thrombosed blood in polyacrylamide in order to more closely simulate a clinical situation.

Characterization Using ex vivo Skin

After conducting experiments on tissue phantoms, testing will be performed using excised pig skin. We will use a standard burn protocol using a 1 cm diameter brass rod (weight 313 g) heated to 100° C. to induce thermal coagulation of blood in the skin. The rod will be applied to the skin samples for 5, 30, 60, and 120 seconds to induce burns. Each temperature will be performed 10 times for statistical information. These times correspond to burn depths from about 100 μm to several millimeters.

Samples from the skins will be stained with hematoxylin and eosin (H&E) and then examined microscopically. Various degrees of thermal damage will be determined by the appearance of cellular structure and collagen. Separation of skin layers, including stratum corneum from the epidermis and epidermis from dermis indicated further degrees of burn injury.

### Characterization on Live Animals

After the ex vivo skin experiment, experiments with live animals will be conducted. Specifically, specimens from rattus norvegicus of the Sprague-Dawley strain will be used for burn depth experiments. The rats would be weighed, anesthetized with ketamine hydrochloride (87 mg/kg, IP) and xylazine (13 mg/kg, IP), backs shaved and cleaned with a surgical scrub, with burns created by contacting the skin with the end of a 1 cm diameter brass rod (weight 313 g), heated to 75° C. using a water bath. Burn severity will be a function of the duration of exposure (5, 10, 20, or 30 sec). Approximately 10 minutes later, a 200 μm thick gel will be placed on the burn area and the burn depth profiling conducted. Burn biopsies will be taken of each burned area and on unburned areas as controls approximately 2 hours after injury, followed by euthanasia of the animals. Biopsies will be sectioned and stained with hematoxylin and eosin (H&E) and then examined microscopically. Various degrees of thermal damage will be determined by the appearance of cellular structure and collagen. Separation of skin layers, including stratum corneum from the epidermis and epidermis from dermis indicated further degrees of burn injury.

### Characterization on Human Patients

A five month study on five burn patients located at the University of Missouri Hospital Burn Center is planned. This process will be a prospective trial in which the tester will be blinded to the origin of the sample. The samples will be coded by research personnel and recorded in a log. Photoacoustic data will not be used to influence treatment decisions during this trial. All work with human subjects will comply with the Institutional Review Board at the University of Missouri. Photoacoustic data as described above will be developed and compared to histological samples of punch biopsies taken from the patients prior to excision. The photoacoustic data and histological burn depth will be compared in the same way as the ex vivo skin samples.

Having illustrated and described the principles of the present invention, it should be apparent to persons skilled in the art that the invention can be modified in arrangement and detail without departing from such principles.

Although the materials and methods of this invention have been described in terms of various embodiments and illustrative examples, it will be apparent to those of skill in the art that variations can be applied to the materials and methods described herein without departing from the concept, spirit and scope of the invention. All such similar substitutes and modifications apparent to those skilled in the art are deemed to be within the spirit, scope and concept of the invention as defined by the appended claims.

1. An apparatus for depth profiling the structure of a subsurface region of skin, the apparatus comprising:
(a) at least one light source for generating a light pulse at a wavelength and intensity effective to generate photoacoustic responses in (i) an absorbing target and (ii) the subsurface region of the skin;
(b) at least one optic fiber coupled to the at least one light source for delivering the light pulse to the absorbing target and the skin;
(c) a probe that comprises (i) a housing comprising an exterior surface, wherein the exterior surface at least a portion of which is placed in contact with the skin, (ii) an inner chamber disposed within the housing and defined, at least in part, by an interior surface of the housing opposite the portion of the exterior surface placed in contact with the skin, (iii) a terminus of the at least one optic fiber located such that it is within or it defines, at least in part, the inner chamber, (iv) the absorbing target, which is located proximate to or in contact with said interior surface of the inner chamber, wherein the photoacoustic response of the absorbing target produces ultrasonic waves, at least a portion of which propagate into and are reflected by the skin, and (v) an acoustic detector for receiving and generating electrical signals in response to the reflected ultrasonic waves and photoacoustic waves generated by the photoacoustic response in the subsurface region of the skin, wherein the acoustic detector is within or defines, at least in part, the inner chamber and is spaced apart from said interior surface of the inner chamber to provide an acoustic delay greater than a delay of electrical noise arising from the light pulse to prevent contamination of said electrical signals; and
(d) at least one circuit coupled to the acoustic detector for processing said electrical signals, wherein the electrical signals generated in response to the reflected ultrasonic waves are interpretable as sound speed in the skin and the electrical signals generated in response to the photoacoustic waves are interpretable, in conjunction with the sound speed, as a depth profile of the structure of the subsurface region of the skin.

2. The apparatus of claim 1, wherein the skin is burned, said at least one circuit identifies signatures of burn damage in the subsurface region of the skin.

3. The apparatus of claim 1, wherein the at least one optic fiber comprises a plurality of optic fibers, each for simultaneously delivering a light pulse to the skin at a single spot.

4. The apparatus of claim 1, wherein the at least one light source comprises a plurality of light sources for generating a corresponding plurality of light pulses in a corresponding plurality of wavelengths and intensities effective to generate photoacoustic responses in the subsurface region of the skin.

5. The apparatus of claim 1, wherein the at least one light source generates two selected wavelengths delivered by the at least one optic fiber to generate different photoacoustic responses in undamaged, reversibly damaged, and necrotic subsurface regions of the skin.

6. (canceled)

7. A method of depth profiling the structure of a subsurface region of skin, the method comprising:
(a) generating a light pulse from at least one light source and directing the light pulse through at least one optic fiber coupled to the at least one light source to deliver the light pulse to and generate photoacoustic responses in:
(i) an absorbing target producing ultrasonic waves, at least a portion of which propagate into and are reflected by the skin; and
(ii) the subsurface region of the skin producing photoacoustic waves in the subsurface region of the skin;
(b) generating electrical signals with an acoustic detector in response to the reflected ultrasonic waves and the photoacoustic waves, wherein the acoustic detector is spaced sufficiently away from the skin to provide an acoustic delay greater than a delay of electrical noise arising from the light pulse to prevent contamination of said electrical signals; and
(c) processing the electrical signals with at least one circuit coupled to the acoustic detector, wherein the electrical signals generated in response to the reflected ultrasonic waves are interpreted to determine a sound speed in the skin and the electrical signals generated in response to the photoacoustic waves are interpreted, in conjunction with the sound speed, to determine a depth profile of the structure of the subsurface region of the skin.

8. The method of claim 7, wherein the skin is burned and the depth profile of the structure of the subsurface region of the skin identifies signatures of burn damage in the subsurface region of the skin.

9. The method of claim 7, wherein the at least one optic fiber coupled to the at least one light source comprises a plurality of optic fibers, each for simultaneously delivering the light pulse from the at least one light source.

10. The method of claim 7, wherein the at least one light source comprises a plurality of light sources for generating a corresponding plurality of light pulses in a corresponding plurality of wavelengths and intensities to generate photoacoustic responses in the subsurface region of the skin.

11. The method of claim 7, wherein the at least one light source generates two selected wavelengths delivered by the at least one optic fiber to generate different photoacoustic responses in undamaged, reversibly damaged, and necrotic subsurface regions of the skin.
